(19) United States
(12) Patent Application Publication
(10) Pub. No.: US 2006/0058736 A1
(43) Pub. Date: Mar. 16, 2006

(54) Novel vaccine

(76) Inventors: Paul G. Alchas, Wayne, NJ (US); Nathalie Garcon, Rixensart (BE); Moncef M Slaoui, Hoverford, PA (US); Christian Van Hoecke, Rixensart (BE)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(21) Appl. No.: 10/476,331

(22) PCT Filed: Apr. 5, 2002
(86) PCT No.: PCT/US02/10938

§ 371 Date: Jul. 20, 2004

Related U.S. Application Data
(60) Provisional application No. 60/286,821, filed on Apr. 27, 2001.

(51) Int. Cl. A61M 5/00

(52) U.S. Cl. 604/117

(57) ABSTRACT

The present invention relates to intradermal delivery of influenza vaccines, specific influenza formulations and methods for preparing and using them.

This invention relates to intradermal delivery of influenza vaccines, specific influenza formulations and methods for preparing and using them.

Influenza virus is one of the most ubiquitous viruses present in the world, affecting both humans and livestock. The influenza virus is an RNA enveloped virus with a particle size of about 125 nm in diameter. It consists basically of an internal nucleocapsid or core of ribonucleic acid (RNA) associated with nucleoprotein, surrounded by a viral envelope with a lipid bilayer structure and external glycoproteins. The inner layer of the viral envelope is composed predominantly of matrix proteins and the outer layer mostly of the host-derived lipid material. The surface glycoproteins neuraminidase (NA) and haemagglutinin (HA) appear as spikes, 10 to 12 nm long, at the surface of the particles. It is these surface proteins, particularly the haemagglutinin, that determine the antigenic specificity of the influenza subtypes.

Typical influenza epidemics cause increases in incidence of pneumonia and lower respiratory disease as witnessed by increased rates of hospitalisation or mortality. The elderly or those with underlying chronic diseases are most likely to experience such complications, but young infants also may suffer severe disease. These groups in particular therefore need to be protected.

Currently available influenza vaccines are either inactivated or live attenuated influenza vaccines. Inactivated flu vaccines comprise one of three types of antigen preparation: inactivated whole virus, sub-virions where purified virus particles are disrupted with detergents or other reagents to solubilise the lipid envelope (so-called “split” vaccine) or purified HA and NA (subunit vaccine). These inactivated vaccines are generally given intramuscularly (i.m.).

Influenza vaccines are usually trivalent vaccines. They generally contain antigens derived from two influenza A virus strains and one influenza B strain. A standard 0.5 ml injectable dose in most cases contains 15 g of haemagglutinin antigen component from each strain, as measured by single radial immunodiffusion (SRD) (J. M. Wood et al.: An improved single radial immunodiffusion technique for the assay of influenza haemagglutinin antigen: adaptation for potency determination of inactivated whole virus and subunit vaccines. J. Biol. Stand. 5 (1977) 237-247; J. M. Wood et al., International collaborative study of single radial diffusion and immunoelectrophoresis techniques for the assay of haemagglutinin antigen of influenza virus. J. Biol. Stand. 9 (1981) 317-330). The influenza virus strains to be incorporated into influenza vaccine each season are determined by the World Health Organisation in collaboration with national health authorities and vaccine manufacturers.

Conventional i.m split or subunit influenza vaccines are prepared by disrupting the virus particle, generally with an organic solvent or a detergent, and separating or purifying the viral proteins to varying extents. Split vaccines are prepared by fragmentation of whole influenza virus, either infectious or inactivated, with solubilizing concentrations of organic solvents or detergents and subsequent removal of the solubilizing agent and some or most of the viral lipid material. Split vaccines generally contain matrix protein and nucleoprotein and sometimes lipid, as well as the membrane envelope proteins. Split vaccines will usually contain most or all of the virus structural proteins although not necessarily in the same proportions as they occur in the whole virus. Subunit vaccines on the other hand consist essentially of highly purified viral surface proteins, haemagglutinin and neuraminidase, which are the surface proteins responsible for eliciting the desired virus neutralising antibodies upon vaccination. Matrix and nucleoproteins are either not detectable or barely detectable in subunit vaccines.

Standards are applied internationally to measure the efficacy of influenza vaccines. The European Union official criteria for an effective vaccine against influenza are set out in the table below. Theoretically, to meet the European Union requirements, and thus be approved for sale in the EU, an influenza vaccine has to meet one of the criteria in the table, for all strains of influenza included in the vaccine. However in practice, at least two or more probably all three of the criteria will need to be met for all strains, particularly for a new vaccine coming onto he market. Under some circumstances two criteria may be sufficient. For example, it may be acceptable for two of the three criteria to be met by all strains while the third criterion is met by some but not all strains (e.g. two out of three strains). The requirements are different for adult populations (18-60 years) and elderly populations (>60 years).

| | 18-60 years | >60 years |
|---|---|---|
| Seroconversion rate* | >40% | >30% |
| Conversion factor** | >2.5 | >2.0 |
| Protection rate*** | >70% | >60% |

*Seroconversion rate is defined as the percentage of vaccinees who have at least a 4-fold increase in serum haemagglutinin inhibition (HI) titres after vaccination, for each vaccine strain.
**Conversion factor is defined as the fold increase in serum HI geometric mean titres (GMTs) after vaccination, for each vaccine strain.
***Protection rate is defined as the percentage of vaccinees with a serum HI titre equal to or greater than 1:40 after vaccination (for each vaccine strain) and is normally accepted as indicating protection.

Current efforts to control the morbidity and mortality associated with yearly epidemics of influenza are based on the use of intramuscularly administered inactivated split or subunit influenza vaccines. The efficacy of such vaccines in preventing respiratory disease and influenza complications ranges from 75% in healthy adults to less than 50% in the elderly.

It would be desirable to provide an alternative way of administering influenza vaccines, in particular a way that is pain-free or less painful than i.m. injection, does not have the same risk of injection site infection, and does not involve the associated negative affect on patient compliance because of “needle fear”. Furthermore, it would be desirable to administer via an administration route that does not have negative effects on the health care worker, such as high risk of needle stick injury.

Experimental intradermal exposure of humans to inactivated influenza vaccines dates back as far as the 1940s. Although the benefits of intradermal vaccination have long been recognised, the success of these vaccinations has been variable and, to date, there is no consensus view that regular vaccination for influenza would be effective and practicable via the intradermal route. Most commonly this variability is associated with the difficulty in getting reproducible vaccine administration into the dermis. Commonly the administration of the vaccine is too deep into the skin causing subcutaneous or intramuscular administration, or too shallow, causing leakage of the vaccine out of the injection site resulting in little or no protection being conferred.

The conventional technique of intradermal injection, the mantoux procedure, is complex and requires a trained and skilled technician to perform. The process comprises steps of cleaning the skin, and then stretching with one hand, and with the bevel of a narrow gauge needle (26-31 gauge) facing upwards the needle is inserted at an angle of between 10-15°. Once the bevel of the needle is inserted, the barrel of the needle is lowered and further advanced whilst providing a slight pressure to elevate it under the skin. The liquid is then injected very slowly thereby forming a bleb or bump on the skin surface, followed by the slow withdrawal of the needle.

Devices have been proposed for providing intradermal injections, which include shortened needles compared to conventional needle sizes. The smaller needles are not intended to penetrate beyond the dermis layer of the individual. Such devices are shown in U.S. Pat. No. 5,527,288, which issued on Jun. 18, 1996; U.S. Pat. No. 4,886,499, which issued on Dec. 12 1989; and U.S. Pat. No. 5,328,483, which issued on July 12, 1994. The proposed devices, however, are not without shortcomings and drawbacks.

For example, the devices shown in U.S. Pat. Nos. 5,527,288 and 4,886,499 are highly specialised injectors. The designs for these injectors include relatively complex arrangements of components that cannot be economically manufactured on a mass production scale. Therefore, such device have limited applicability and use.

Similarly, the device shown in U.S. Pat. No. 5,328,483 requires a specially designed injector and, therefore, is not readily adapted to be used with a variety of syringe types. Additionally, the assembly of that patent is not conducive to economical mass production.

Examples of intradermal influenza vaccination via the Mantoux technique or jet gun injectors include: Crowe (1965) Am J Medical Technology 31, 387-396; McElroy (1969) in New Eng J of Medicine, 6 November, page 1076;Tauraso et al (1969) Bull Wld Hlth Org 41, 507-516; Foy (1970) in a letter to JAMA, Jun. 7, 1970, vol 213 page 130; letter to the British Medical Journal, Oct. 29, 1977 page 1152; Brooks et al (1977) Annals of Allergy 39, 110-112; Brown et al (1977) J Infectious Disease 136, 466-471; Halperin et al (1979) AJPH 89, 1247-1252; Herbert and Larke (1979) J Infectious Diseases 140, 234-238; Bader (1980) in a letter to AJPH, vol. 70 no. 5; Niculescu et al (1981) in Arch Roum Path Exp Microbiol, 40, 67-70.

Thus, the literature shows an interest in intradermal vaccination between the mid-sixties (or earlier) and the early 1980s. However, the prevailing view appears to have been that two doses of vaccine would be needed. Also, there was a widely held view that due to the difficulty of administration and the lack of certainty that the low volume of vaccine would successfully be located in the desired region, the use of the intradermal delivery route has not been considered for conventional mass vaccination purposes.

Thus, the commercially available influenza vaccines remain the intramuscularly administered split or subunit injectable vaccines.

Although intradermal flu vaccines based on inactivated virus have been studied in previous years, the fact that no intradermal flu vaccine is currently on the market reflects the difficulty to achieve effective vaccination via this route.

It has now been discovered that certain influenza vaccines, make particularly good intradermal vaccines when administered reliably into the dermis of the patient by a specific delivery device. In particular, an intradermal administration of such an influenza virus vaccine preparation in this manner stimulates systemic immunity at a protective level with a low dose of antigen. Furthermore, the international criteria for an effective flu vaccine are met. More specifically, intradermal administration of the low antigen dose vaccine can produce a systemic seroconversion (4-fold increase in anti-HA titres) equivalent to that obtained by s.c. administration of the same vaccine.

As used herein, the term “intradermal delivery” means delivery of the vaccine to the region of the dermis in the skin. However, the vaccine will not necessarily be located exclusively in the dermis. The dermis is the layer in the skin located between about 0.5 and about 3 mm from the surface in human skin, but there is a certain amount of variation between individuals and in different parts of the body. In general, it can be expected to reach the dermis by going 1.5 mm below the surface of the skin. The dermis is located between the stratum corneum and the epidermis at the surface and the subcutaneous layer below.

Depending on the mode of delivery, the vaccine may ultimately be located solely or primarily within the dermis, or it may ultimately be distributed within the epidermis and the dermis.

Accordingly, in a first aspect, the present invention provides an intradermal delivery device for the intradermal delivery of a flu vaccine, the device comprising:

i a container having a reservoir comprising a flu vaccine and having an outlet port that allows the flu vaccine to exit the reservoir during an injection;

ii a needle in fluid communication with the outlet port, the needle having a forward end that is adapted to penetrate the skin of an animal; and

iii a limiter that surrounds the needle and has a skin engaging surface that is adapted to be placed against the skin of an animal to receive an intradermal injection, the needle forward end extending beyond the skin engaging surface a selected distance such that the limiter limits an amount that the needle forward end penetrates the skin.

Delivery of a flu vaccine using such an intradermal delivery device is highly effective and reproducible, and reliably provokes an effective protective response using a fraction of the vaccine that would otherwise be required through i.m. delivery.

Flu Preferred Features

Preferably the flu vaccine comprises a non live influenza antigen preparation. Preferably the non-live antigen preparation is a split influenza preparation or a subunit antigen preparation prepared from live virus. Most preferably the antigen is a split influenza antigen preparation. The split influenza antigen preparation may be produced according to the methods described herein.

Preferably the vaccine is a one-dose influenza vaccine for intradermal delivery. The influenza antigen preparation may be produced according to a variety of known methods, including in particular methods described herein.

Preferably the vaccine is a trivalent vaccine.

The vaccine according to the invention meets some or all of the EU criteria for influenza vaccines as set out hereinabove, such that the vaccine is approvable in Europe. Preferably, at least two out of the three EU criteria are met, for the or all strains of influenza represented in the vaccine. More preferably, at least two criteria are met for all strains and the third criterion is met by all strains or at least by all but one of the strains. Most preferably, all strains present meet all three of the criteria.

The vaccine according to the invention may have a lower quantity of haemagglutinin than conventional vaccines and is administered in a lower volume. Preferably the quantity of haemagglutinin per strain of influenza is about 1-7.5 μg, more preferably approximately 3 μg or approximately 5 μg, which is about one fifth or one third, respectively, of the dose of haemagglutinin used in conventional vaccines for intramuscular administration. Preferably the volume of a dose of vaccine according to the invention is between 0.025 ml and 2.5 ml, more preferably approximately 0.1 ml or approximately 0.2 ml. A 50 μl dose volume might also be considered. A 0.1 ml dose is approximately one fifth of the volume of a conventional intramuscular flu vaccine dose. The volume of liquid that can be administered intradermally depends in part upon the site of the injection. For example, for an injection in the deltoid region, 0.1 ml is the maximum preferred volume whereas in the lumbar region a large volume e.g. about 0.2 ml can be given.

Preferably the spilt flu vaccine is obtainable by the following process:

(i) harvesting of virus-containing material from a culture;

(ii) clarification of the harvested material to remove non-virus material;

(iii) concentration of the harvested virus;

(iv) a further step to separate whole virus from non-virus material;

(v) splitting of the whole virus using a suitable splitting agent in a density gradient centrifugation step;

(vi) filtration to remove undesired materials; wherein the steps are performed in that order but not necessarily consecutively.

Preferably the virus is grown on eggs, more particularly on embryonated hen eggs, in which case the harvested material is allantoic fluid.

Preferably the clarification step is performed by centrifugation at a moderate speed. Alternatively a filtration step may be used for example with a 0.2 μm membrane. The clarification step gets rid of the bulk of the culture-derived e.g. egg-derived material.

Preferably the concentration step employs an adsorption method, most preferably using CaHPO₄ . Alternatively filtration may be used, for example ultrafiltration.

Preferably the further separation step (iv) is a zonal centrifugation separation, particularly one using a sucrose gradient. Optionally the gradient contains a preservative to prevent microbial growth.

Preferably the splitting step is performed in a further sucrose gradient, wherein the sucrose gradient contains the splitting agent.

Preferably the filtration step (vi) is an ultrafiltration step which concentrates the split virus material.

Preferably there is at least one sterile filtration step, optionally at the end of the process.

Optionally there is an inactivation step prior to the final filtration step.

Preferably the intradermal vaccines described herein comprise at least one non-ionic surfactant.

Preferably the vaccines according to the invention are administered to a location between about 1.0 and 2.0 mm below the surface of the skin. More preferably the vaccine is delivered to a distance of about 1.5 mm below the surface of the skin.

The vaccine to which the invention relates is a split virion vaccine comprising particles. Preferably the vaccine contains particles having a mean particle size below 200 nm, more preferably between 50 and 180 nm, most preferably between 100 and 150 nm, as measured using a dynamic light scattering method (Malvern Zeta Sizer). Particle size may vary from season to season depending on the strains.

The split influenza virus antigen preparation used in the present invention preferably contains at least one non-ionic surfactant. Preferably the non-ionic surfactant is at least one surfactant selected from the group consisting of the octyl- or nonylphenoxy polyoxyethanols (for example the commercially available Triton™ series), polyoxyethylene sorbitan esters (Tween™ series) and polyoxyethylene ethers or esters of general formula (I):
HO(CH₂CH₂O)ₙ-A-R (I)
wherein n is 1-50, A is a bond or —C(O)—, R is C₁₋₅₀ alkyl or phenyl C₁₋₅₀ alkyl; and combinations of two or more of these.

Preferred is a combination of two non-ionic surfactants, one from each of the octylphenoxy polyoxyethanols and the polyoxyethylene sorbitan esters, in particular a combination of Tween 80 and Triton X-100. Further possible and preferred combinations of detergents are discussed hereinbelow.

Preferred surfactants falling within formula (I) are molecules in which n is 4-24, more preferably 6-12, and most preferably 9; the R component is C₁₋₅₀, preferably C₄-C₂₀alkyl and most preferably C₁₂ alkyl.

Octylphenoxy polyoxyethanols and polyoxyethylene sorbitan esters are described in “Surfactant systems” Eds: Attwood and Florence (1983, Chapman and Hall). Octylphenoxy polyoxyethanols (the octoxynols), including t- octylphenoxypolyethoxyethanol (Triton X-100™) are also described in Merck Index Entry 6858 (Page 1162, 12^{th} Edition, Merck & Co. Inc., Whitehouse Station, N.J., USA; ISBN 0911910-12-3). The polyoxyethylene sorbitan esters, including polyoxyethylene sorbitan monooleate (Tween 80™) are described in Merck Index Entry 7742 (Page 1308, 12^{th} Edition, Merck & Co. Inc., Whitehouse Station, N.J., USA; ISBN 0911910-12-3). Both may be manufactured using methods described therein, or purchased from commercial sources such as Sigma Inc.

Particularly preferred non-ionic surfactants include Triton X-45, t-octylphenoxy polyethoxyethanol (Triton X-100), Triton X-102, Triton X-114, Triton X-165, Triton X-205, Triton X-305, Triton N-57, Triton N-101, Triton N-128, Breij 35, polyoxyethylene-9-lauryl ether (laureth 9) and polyoxyethylene-9-stearyl ether (steareth 9). Triton X-100 and laureth 9 are particularly preferred. Also particularly preferred is the polyoxyethylene sorbitan ester, polyoxyethylene sorbitan monooleate (Tween 80™).

Further suitable polyoxyethylene ethers of general formula (I) are selected from the following group: polyoxyethylene-8-stearyl ether, polyoxyethylene4-lauryl ether, polyoxyethylene-35-lauryl ether, and polyoxyethylene-23-lauryl ether.

Alternative terms or names for polyoxyethylene lauryl ether are disclosed in the CAS registry. The CAS registry number of polyoxyethylene-9 lauryl ether is: 9002-92-0. Polyoxyethylene ethers such as polyoxyethylene lauryl ether are described in the Merck index (12^{th} ed: entry 7717, Merck & Co. Inc., Whitehouse Station, N.J., USA; ISBN 0911910-12-3). Laureth 9 is formed by reacting ethylene oxide with dodecyl alcohol, and has an average of nine ethylene oxide units.

The ratio of the length of the polyoxyethylene section to the length of the alkyl chain in the surfactant (i.e. the ratio of n: alkyl chain length), affects the solubility of this class of surfactant in an aqueous medium. Thus, the surfactants of the present invention may be in solution or may form particulate structures such as micelles or vesicles. As a solution, the surfactants of the present invention are safe, easily sterilisable, simple to administer, and may be manufactured in a simple fashion without the GMP and QC issues associated with the formation of uniform particulate structures. Some polyoxyethylene ethers, such as laureth 9, are capable of forming non-vesicular solutions. However, polyoxyethylene-8 palmitoyl ether (C₁₈E₈) is capable of forming vesicles. Accordingly, vesicles of polyoxyethylene-8 palmitoyl ether in combination with at least one additional non-ionic surfactant, can be employed in the formulations of the present invention.

Preferably, the polyoxyethylene ether used in the formulations of the present invention has haemolytic activity. The haemolytic activity of a polyoxyethylene ether may be measured in vitro, with reference to the following assay, and is as expressed as the highest concentration of the surfactant which fails to cause lysis of the red blood cells:

1. Fresh blood from guinea pigs is washed with phosphate buffered saline (PBS) 3 times in a desk-top centrifuge. After re-suspension to the original volume the blood is further diluted 10 fold in PBS.

2. 50 μl of this blood suspension is added to 800 μl of PBS containing two-fold dilutions of detergent.

3. After 8 hours the haemolysis is assessed visually or by measuring the optical density of the supernatant. The presence of a red supernatant, which absorbs light at 570 nm indicates the presence of haemolysis.

4. The results are expressed as the concentration of the first detergent dilution at which hemolysis no longer occurs.

Within the inherent experimental variability of such a biological assay, the polyoxyethylene ethers, or surfactants of general formula (I), of the present invention preferably have a haemolytic activity, of approximately between 0.5-0.0001%, more preferably between 0.05-0.0001%, even more preferably between 0.005-0.0001%, and most preferably between 0.003-0.0004%. Ideally, said polyoxyethylene ethers or esters should have a haemolytic activity similar (i.e. within a ten-fold difference) to that of either polyoxyethylene-9 lauryl ether or polyoxyethylene-8 stearyl ether.

Two or more non-ionic surfactants from the different groups of surfactants described may be present in the vaccine formulation described herein. In particular, a combination of a polyoxyethylene sorbitan ester such as polyoxyethylene sorbitan monooleate (Tween 80™) and an octoxynol such as t-octylphenoxypolyethoxyethanol (Triton) X-₁₀₀™ is preferred. Another particularly preferred combination of non-ionic surfactants comprises laureth 9 plus a polyoxyethylene sorbitan ester or an octoxynol or both.

Preferably the or each non-ionic surfactant is present in the final vaccine formulation at a concentration of between 0.001 to 20%, more preferably 0.01 to 10%, and most preferably up to about 2% (w/v). Where one or two surfactants are present, these are generally present in the final formulation at a concentration of up to about 2% each, typically at a concentration of up to about 0.6% each. One or more additional surfactants may be present, generally up to a concentration of about 1 % each and typically in traces up to about 0.2% or 0.1% each. Any mixture of surfactants may be present in the vaccine formulations according to the invention.

Non-ionic surfactants such as those discussed above have preferred concentrations in the final vaccine composition as follows: polyoxyethylene sorbitan esters such as Tween 80™: 0.01 to 1%, most preferably about 0.1% (w/v); octyl- or nonylphenoxy polyoxyethanols such as Triton X-100™ or other detergents in the Triton series: 0.001 to 0.1%, most preferably 0.005 to 0.02% (w/v); polyoxyethylene ethers of general formula (I) such as laureth 9: 0.1 to 20%, preferably 0.1 to 10% and most preferably 0.1 to I % or about 0.5% (w/v).

Other reagents may also be present in the formulation. As such the formulations of the present invention may also comprise a bile acid or a derivative thereof, in particular in the form of a salt. These include derivatives of cholic acid and salts thereof, in particular sodium salts of cholic acid or cholic acid derivatives. Examples of bile acids and derivatives thereof include cholic acid, deoxycholic acid, chenodeoxycholic acid, lithocholic acid, ursodeoxycholic acid, hyodeoxycholic acid and derivatives such as glyco-, tauro-, amidopropyl-1-propanesulfonic-, amidopropyl-2-hydroxy-1-propanesulfonic derivatives of the aforementioned bile acids, or N,N-bis (3Dgluconoamidopropyl) deoxycholamide. A particularly preferred example is sodium deoxycholate (NaDOC) which may be present in the final vaccine dose.

The vaccine formulation according to the invention preferably comprises a split flu virus preparation in combination with one or more non-ionic surfactants. The one or more non-ionic surfactants may be residual from the process by which the split flu antigen preparation is produced, and/or added to the antigen preparation later. The concentration of the or each non-ionic surfactant may be adjusted to the desired level at the end of the splitting/purification process. It is believed that the split flu antigen material may be stabilised in the presence of a non-ionic surfactant, though it will be understood that the invention does not depend upon this necessarily being the case.

The vaccine according to the invention may further comprise an adjuvant or immunostimulant such as but not limited to detoxified lipid A from any source and non-toxic derivatives of lipid A, saponins and other reagents capable of stimulating a TH1 type response.

It has long been known that enterobacterial lipopolysaccharide (LPS) is a potent stimulator of the immune system, although its use in adjuvants has been curtailed by its toxic effects. A non-toxic derivative of LPS, monophosphoryl lipid A (MPL), produced by removal of the core carbohydrate group and the phosphate from the reducing-end glucosamine, has been described by Ribi et al (1986, Immunology and Immunopharmacology of bacterial endotoxins, Plenum Publ. Corp., NY, p407-419) and has the following structure:

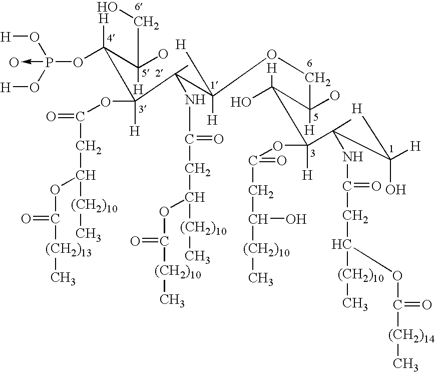

A further detoxified version of MPL results from the removal of the acyl chain from the 3-position of the disaccharide backbone, and is called 3-0-Deacylated monophosphoryl lipid A (3D-MPL). It can be purified and prepared by the methods taught in GB 2122204B, which reference also discloses the preparation of diphosphoryl lipid A, and 3-0-deacylated variants thereof.

A preferred form of 3D-MPL is in the form of an emulsion having a small particle size less than 0.2μm in diameter, and its method of manufacture is disclosed in WO 94/21292. Aqueous formulations comprising monophosphoryl lipid A and a surfactant have been described in WO9843670A2.

The bacterial lipopolysaccharide derived adjuvants to be formulated in the compositions of the present invention may be purified and processed from bacterial sources, or alternatively they may be synthetic. For example, purified monophosphoryl lipid A is described in Ribi et al 1986 (supra), and 3-O-Deacylated monophosphoryl or diphosphoryl lipid A derived from Salmonella sp. is described in GB 2220211 and U.S. Pat. No. 4,912,094. Other purified and synthetic lipopolysaccharides have been described (Hilgers et al., 1986, Int.Arch.Allergy.Immunol., 79(4):392-6; Hilgers et al., 1987, Immunology, 60(1):141-6; and EP 0 549 074 B 1). A particularly preferred bacterial lipopolysaccharide adjuvant is 3D-MPL.

Accordingly, the LPS derivatives that may be used in the present invention are those immunostimulants that are similar in structure to that of LPS or MPL or 3D-MPL. In another aspect of the present invention the LPS derivatives may be an acylated monosaccharide, which is a sub-portion to the above structure of MPL.

Saponins are taught in: Lacaille-Dubois, M and Wagner H. (1996. A review of the biological and pharmacological activities of saponins. Phytomedicine vol 2 pp 363-386). Saponins are steroid or triterpene glycosides widely distributed in the plant and marine animal kingdoms. Saponins are noted for forming colloidal solutions in water which foam on shaking, and for precipitating cholesterol. When saponins are near cell membranes they create pore-like structures in the membrane which cause the membrane to burst. Haemolysis of erythrocytes is an example of this phenomenon, which is a property of certain, but not all, saponins.

Saponins are known as adjuvants in vaccines for systemic administration. The adjuvant and haemolytic activity of individual saponins has been extensively studied in the art (Lacaille-Dubois and Wagner, supra). For example, Quil A (derived from the bark of the South American tree Quillaja Saponaria Molina), and fractions thereof, are described in U.S. Pat. No. 5,057,540 and “Saponins as vaccine adjuvants”, Kensil, C. R., Crit Rev Ther Drug Carrier Syst, 1996, 12 (1-2):1-55; and EP 0 362 279 B 1. Particulate structures, termed Immune Stimulating Complexes (ISCOMS), comprising fractions of Quil A are haemolytic and have been used in the manufacture of vaccines (Morein, B., EP 0 109 942 B 1; WO 96/11711; WO 96/33739). The haemolytic saponins QS21 and QS 17 (HPLC purified fractions of Quil A) have been described as potent systemic adjuvants, and the method of their production is disclosed in US Patent No.5,057,540 and EP 0 362 279 B 1. Other saponins which have been used in systemic vaccination studies include those derived from other plant species such as Gypsophila and Saponaria (Bomford et al., Vaccine, 10(9):572-577, 1992).

An enhanced system involves the combination of a non-toxic lipid A derivative and a saponin derivative particularly the combination of QS21 and 3D-MPL as disclosed in WO 94/00153, or a less reactogenic composition where the QS21 is quenched with cholesterol as disclosed in WO 96/33739.

A particularly potent adjuvant formulation involving QS21 and 3D-MPL in an oil in water emulsion is described in WO 95/17210 and is a preferred formulation.

Accordingly in one embodiment of the present invention there is provided a vaccine comprising an influenza antigen preparation of the present invention adjuvanted with detoxified lipid A or a non-toxic derivative of lipid A, more preferably adjuvanted with a monophosphoryl lipid A or derivative thereof.

Preferably the vaccine additionally comprises a saponin, more preferably QS21.

Preferably the formulation additionally comprises an oil in water emulsion. The present invention also provides a method for producing a vaccine formulation comprising mixing an antigen preparation of the present invention together with a pharmaceutically acceptable excipient, such as 3D-MPL.

Additional components that are preferably present in an adjuvanted vaccine formulation according to the invention include non-ionic detergents such as the octoxynols and polyoxyethylene esters as described herein, particularly t-octylphenoxy polyethoxyethanol (Triton X-100) and polyoxyethylene sorbitan monooleate (Tween 80); and bile salts or cholic acid derivatives as described herein, in particular sodium deoxycholate or taurodeoxycholate. Thus, a particularly preferred formulation comprises 3D-MPL, Triton X-100, Tween 80 and sodium deoxycholate, which may be combined with an influenza virus antigen preparation to provide a vaccine suitable for intradermal application.

In one preferred embodiment of the present invention, the intradermal influenza vaccines comprise a vesicular adjuvant formulation comprising cholesterol, a saponin and an LPS derivative. In this regard the preferred adjuvant formulation comprises a unilamellar vesicle comprising cholesterol, having a lipid bilayer preferably comprising dioleoyl phosphatidyl choline, wherein the saponin and the LPS derivative are associated with, or embedded within, the lipid bilayer. More preferably, these adjuvant formulations comprise QS21 as the saponin, and 3D-MPL as the LPS derivative, wherein the ratio of QS21: cholesterol is from 1:1 to 1:100 weight/weight, and most preferably 1:5 weight/weight. Such adjuvant formulations are described in EP 0 822 831 B, the disclosure of which is incorporated herein by reference.

The invention also provides a method for the prophylaxis of influenza infection or disease in a subject which method comprises administering to the subject intradermally a split influenza vaccine according to the invention.

The invention provides in a further aspect a pharmaceutical kit comprising an intradermal administration device and a vaccine formulation as described herein. The device is preferably supplied already filled with the vaccine. Preferably the vaccine is in a liquid volume smaller than for conventional intramuscular vaccines as described herein, particularly a volume of between about 0.05 ml and 0.2 ml.

The influenza vaccine according to the invention is preferably a multivalent influenza vaccine comprising two or more strains of influenza. Most preferably it is a trivalent vaccine comprising three strains. Conventional influenza vaccines comprise three strains of influenza, two A strains and one B strain. However, monovalent vaccines, which may be useful for example in a pandemic situation, are not excluded from the invention. A monovalent, pandemic flu vaccine will most likely contain influenza antigen from a single A strain.

The influenza virus preparations may be derived from the conventional embryonated egg method, or they may be derived from any of the new generation methods using tissue culture to grow the virus. Suitable cell substrates for growing the virus include for example dog kidney cells such as MDCK or cells from a clone of MDCK, MDCK-like cells, monkey kidney cells such as AGMK cells including Vero cells, or any other mammalian cell type suitable for the production of influenza virus for vaccine purposes. Suitable cell substrates also include human cells e.g. MRC-5 cells. Suitable cell substrates are not limited to cell lines; for example primary cells such as chicken embryo fibroblasts are also included.

Traditionally split flu was produced using a solvent/detergent treatment, such as tri-n-butyl phosphate, or diethylether in combination with Tween™ (known as “Tween-ether” splitting) and this process is still used in some production facilities. Other splitting agents now employed include detergents or proteolytic enzymes or bile salts, for example sodium deoxycholate as described in patent no. DD 155 875, incorporated herein by reference. Detergents that can be used as splitting agents include cationic detergents e.g. cetyl trimethyl ammonium bromide (CTAB), other ionic detergents e.g. laurylsulfate, taurodeoxycholate, or non-ionic detergents such as the ones described above including Triton X-100 (for example in a process described in Lina et al, 2000, Biologicals 28, 95-103) and Triton N-101, or combinations of any two or more detergents.

Further suitable splitting agents which can be used to produce split flu virus preparations include:

1. Bile acids and derivatives thereof including: cholic acid, deoxycholic acid, chenodeoxy colic acid, lithocholic acid ursodeoxycholic acid, hyodeoxycholic acid and derivatives like glyco-, tauro-, amidopropyl-1-propanesulfonic-, amidopropyl-2-hydroxy-1-propanesulfonic derivatives of the aforementioned bile acids, or N,N-bis (3DGluconoamidopropyl) deoxycholamide. A particular example is sodium deoxycholate (NaDOC) which may be present in trace amounts in the final vaccine dose.

2. Alkylglycosides or alkylthioglycosides, where the alkyl chain is between C6-C18 typical between C8 and C14, sugar moiety is any pentose or hexose or combinations thereof with different linkages, like 1→6, 1→5, 1→4, 1→3, 1→2. The alkyl chain can be saturated unsaturated and/or branched.

3. Derivatives of 2 above, where one or more hydroxyl groups, preferably the 6 hydroxyl group is/are modified, like esters, ethoxylates, sulphates, ethers, carbonates, sulphosuccinates, isethionates, ethercarboxylates, quarternary ammonium compounds.

4. Acyl sugars, where the acyl chain is between C6 and C18, typical between C8 and C12, sugar moiety is any pentose or hexose or combinations thereof with different linkages, like 1→6, 1→5, 1→4, 1→3, 1→2. The acyl chain can be saturated or unsaturated and/or branched, cyclic or non-cyclic, with or without one or more heteroatoms e.g. N, S, P or O.

5. Sulphobetaines of the structure R—N,N—(R1 ,R2)-3-amino-1-propanesulfonate, where R is any alkyl chain or arylalkyl chain between C6 and C18, typical between C8 and C16. The alkyl chain R can be saturated, unsaturated and/or branched. R1 and R2 are preferably alkyl chains between C1 and C4, typically C1, or R1, R2 can form a heterocyclic ring together with the nitrogen.

6. Betains of the structure R—N,N—(R1,R2)-glycine, where R is any alkyl chain between C6 and C18, typical between C8 and C16. The alkyl chain can be saturated unsaturated and/or branched. R1 and R2 are preferably alkyl chains between C1 and C4, typically C1, or R1 and R2 can form a heterocyclic ring together with the nitrogen.

7. N,N-dialkyl-glucamides, of the Structure R—(N—R1)-glucamide, where R is any alkylchain between C6 and C18, typical between C8 and C12. The alkyl chain can be saturated unsaturated and/or branched or cyclic. R1, and R2 are alkyl chains between C1 and C6, typically C1. The sugar moiety might be modified with pentoses or hexoses.

8. Quarternary ammonium compounds of the structure R, —N⁺(—R1, —R2, —R3), where R is any alkylchain between C6 and C20, typically C20. The alkyl chain can be saturated unsaturated and/or branched. R1, R2 and R3 are preferably alkyl chains between C1 and C4, typically C1, or R1, R2 can form a heterocyclic ring together with the nitrogen. A particular example is cetyl trimethyl ammonium bromide (CTAB).

The preparation process for a split vaccine will include a number of different filtration and/or other separation steps such as ultracentrifugation, ultrafiltration, zonal centrifugation and chromatography (e.g. ion exchange) steps in a variety of combinations, and optionally an inactivation step eg with formaldehyde or β-propiolactone or U.V. which may be carried out before or after splitting. The splitting process may be carried out as a batch, continuous or semi-continuous process.

Preferably, a bile salt such as sodium deoxycholate is present in trace amounts in a split vaccine formulation according to the invention, preferably at a concentration not greater than 0.05%, or not greater than about 0.01%, more preferably at about 0.0045% (w/v).

Preferred split flu vaccine antigen preparations according to the invention comprise a residual amount of Tween 80 and/or Triton X-100 remaining from the production process, although these may be added or their concentrations adjusted after preparation of the split antigen. Preferably both Tween 80 and Triton X-100 are present. The preferred ranges for the final concentrations of these non-ionic surfactants in the vaccine dose are:

Tween 80: 0.01 to I %, more preferably about 0. 1% (v/v)

Triton X-100: 0.001 to 0.1 (% w/v), more preferably 0.005 to 0.02% (w/v).

The presence of the combination of these two surfactants, in low concentrations, was found to promote the stability of the antigen in solution. It is possible that this enhanced stability rendered the antigen more immunogenic intradermally than previous formulations have been. Such an enhancement could arise from a prevalence of small antigen aggregates or the enhancement of the native conformation of the antigen. It will be appreciated that the invention does not depend upon this theoretical explanation being correct.

In a particular embodiment, the preferred split virus preparation also contains laureth 9, preferably in the range 0.1 to 20%, more preferably 0.1 to 10% and most preferably 0.1 to 1% (w/v).

The vaccines according to the invention generally contain not more than 25% (w/v) of detergent or surfactant, preferably less than 15% and most preferably not more than about 2%.

The invention provides in another aspect a method of manufacturing an influenza vaccine for intradermal application which method comprises:

(i) providing a split influenza virus preparation produced essentially as for a conventional injected (e.g. intramuscular) influenza vaccine and comprising at least one non-ionic surfactant;

(ii) optionally adjusting the concentration of the haemagglutinin and/or the concentration of non-ionic surfactant in the preparation;

(iii) filling an intradermal delivery device with a vaccine dose from the split influenza virus preparation, said dose being a suitable volume for intradermal administration, preferably between about 0.05 ml and 0.2 ml of liquid vaccine.

Preferably the intradermal delivery device is a device as described herein.

A further optional step in the method according to this aspect of the invention includes the addition of an absorption-enhancing surfactant such as laureth 9, and/or the addition of an adjuvant such as a non-toxic lipid A derivative, particularly 3D-MPL.

Processes for producing conventional injected inactivated flu vaccines are well known and described in the literature. Such processes may be modified for producing, eg, a one-dose intradermal vaccine for use in the present invention, for example by the inclusion of a step for adjusting the concentration of other components e.g. non-ionic surfactants to a suitable % (w/v) for an intradermal vaccine according to the invention. However, the active ingredient of the vaccine, i.e. the influenza antigen can be essentially the same for the conventional intramuscular vaccine and the one-dose intradermal vaccines according to the invention.

Preferably, the vaccine formulations according to the invention do not include formulations that do not meet at least two of the EU criteria for all strains, when administered as a one-dose vaccine.

Device

The preferred device of the invention for intradermal delivery comprises a drug container having a flu vaccine, the container being in operative combination with a needle, such that the vaccine in the container can be delivered through the needle as required. The device further comprises a limiter, adapted to limit the extent to which the needle can penetrate the skin, such that vaccine is delivered to the dermis.

The invention also extends to the provision of the device in component form, for example, in which a needle assembly having a limiter device is provided in conjunction with a separate prefilled vaccine container, the container and the needle assembly being attachable to produce a preferred intradermal delivery device. Suitable containers include syringe bodies, and the needle assembly of the present invention is advantageous in that it can be used with a variety of such containers.

Furthermore the invention extends to kits in which the device of the present invention comprising a needle assembly connected to an empty container is supplied in combination with a flu vaccine.

Accordingly the present invention extends to a kit for use in intradermal flu vaccine delivery, the kit comprising:

(a) a vaccine container comprising a flu vaccine; and

(b) a hypodermic needle assembly, the assembly comprising:
i a hub portion that is able to be attached to a drug container;
ii a needle supported by the hub portion, the needle having a hollow body with a forward end extending away from the hub portion; and
iii a limiter portion that surrounds the needle and extends away from the hub portion toward the forward end of the needle, the limiter portion having a skin engaging surface that is adapted to be received against the skin of an animal to receive an intradermal injection, the needle forward end extending beyond the skin engaging surface a selected distance such that the limiter portion limits an amount that the needle is able to penetrate through the skin of an animal.

Preferably the kit comprises a needle assembly and prefilled vaccine container in the form of a syringe body.

The delivery device of the present invention will now be further described in the following, non-limiting Figures and description, wherein:

FIG. 1 is an exploded, perspective illustration of a needle assembly according to this invention.

FIG. 2 is a partial cross-sectional illustration of the embodiment of FIG. 1.

FIG. 3 shows the embodiment of FIG. 2 attached to a syringe body to form an injection device.

FIG. 4 is an exploded, side view of another embodiment of an injection device designed according to this invention.

FIG. 5 is a cross-sectional illustration taken along the lines A-A in FIG. 4 but showing the components in an assembled condition.

FIG. 6 is an exploded, cross-sectional view similar to that shown in FIG. 5 showing an alternative embodiment.

FIG. 7 shows the embodiment of FIG. 6 in an assembled condition.

FIG. 8 is a flow chart diagram that schematically illustrates a method of filling a device according to this invention.

### DETAILED DESCRIPTION OF THE PREFERRED EMBODIMENTS

FIGS. 1 and 2 diagrammatically illustrate the needle assembly 20 of the present invention that is designed to be used for making intradermal injections, FIG. 3 illustrates the drug container such as syringe 60 for use with the needle assembly 20, and FIGS. 4-7 illustrate the intradermal delivery device 80 of the present invention for making intradermal injections. Intradermal injections involve administering vaccines into the skin of an animal such as a human.

The needle assembly 20 includes a hub 22 that supports a needle 24. The limiter receives at least a portion of the hub 22 so that the limiter 26 generally surrounds the needle 24 as best seen in FIG. 2.

One end 30 of the hub 22 is able to be secured to a receiver 32 of a syringe. A variety of syringe types can be used with a needle assembly designed according to this invention, with several examples being given below. The opposite end of the hub 22 preferably includes extensions 34 that are nestingly received against abutment surfaces 36 within the limiter 26. A plurality of ribs 38 preferably are provided on the limiter 26 to provide structural integrity and to facilitate handling the needle assembly 20.

By appropriately designing the size of the components, a distance d between a forward end or tip 40 of the needle 24 and a skin engaging surface 47 on the limiter 26 can be tightly controlled. The distance d preferably is in a range from approximately 0.5 millimetres to approximately 3 millimetres. When the forwarded end 40 of the needle 24 extends beyond the skin engaging surface 42 a distance within that range, an intradermal injection is ensured because the needle is unable to penetrate any further than the typical dermis layer of an animal. Typical tissue layers include an epidermis between 50 and 100 micrometres, a dermis layer between 2 and 3mm then subcutaneous tissue followed by muscle tissue.

As can be best seen in FIG. 2, the limiter 26 includes an opening 44 through which the forward end 40 of the needle 24 protrudes. The dimensional relationship between the opening 44 and the needle 40 can be controlled depending on the needs of a particular situation. In the illustrated embodiment, the skin engaging surface 42 is general planar and continuous and provides a stable placement of the needle assembly 20 against an animal's skin. Although not specifically illustrated, it may be advantageous to have the skin engaging surface be slightly concave or convex in order to facilitate stretching or gathering the animal's skin in the vacinity of the needle tip 40 to facilitate making an injection. Additionally, the ribs 38 may be extended beyond the skin engaging surface 42 to further facilitate manipulating the skin in the vicinity where the injection is to be given.

Regardless of the shape or contour of the skin engaging surface 42, the preferred embodiment includes enough of a surface area that contacts the skin to facilitate stabilising the injector relative to the animal's skin. In the most preferred arrangement, the skin engaging surface 42 facilitates maintaining the injector in a generally perpendicular orientation relative to the skin surface.

It is important to note that although FIGS. 1 and 2 illustrate a two-piece assembly where the hub 22 is made separate from the limiter 26, this invention is not limited to such an arrangement. Forming the hub 22 and limiter 26 integrally from a single piece of plastic material is an alternative to the example shown in FIGS. 1 and 2. Additionally, it is possible to adhesively or otherwise secure the hub 22 to the limiter 26 in the position illustrated in FIG. 2 so that the needle assembly 20 becomes a single piece unit upon assembly.

Having a hub 22 and limiter 26 provides the advantage of making an intradermal needle practical to manufacture. The preferred needle size is a small gauge hypodermic needle, commonly known as a 30 gauge or 31 gauge needle. Having such a small diameter needle presents a challenge to make a needle short enough to prevent undue penetration beyond the dermis layer of an animal. The limiter 26 and the hub 22 facilitate utilising a needle 24 that has an overall length that is much greater than the effective length of the needle, which penetrates the individual's tissue during an injection. With a needle assembly designed according to this invention, manufacturing is enhanced because larger length needles can be handled during the maufacturing and assembly processes while still obtaining the advantages of having a shorter needle for purposes of completing an intradermal injection.

FIG. 3 illustrates a needle assembly 20 secured to a drug container such as a syringe 60. A generally cylindrical syringe body 62 can be made of plastic or glass as is known in the art. The syringe body 62 provides a reservoir 64 for containing a substance to be administered during an injection. A plunger 66 has a manual activation flange 68 at one end with a stopper 70 at an opposite end as known in the art. Manual movement of the plunger 66 through the reservoir 64 forces the substance within the reservoir 64 out of the end 40 of the needle as desired.

The hub 22 can be secured to the syringe body 62 in a variety of known manners. In one example, an interference fit is provided between the interior of the hub 22 and the exterior of the outlet port portion 72 of the syringe body 62. In another example, a conventional luer fit arrangement is provided to secure the hub 22 on the end of the syringe 60. As can be appreciated from FIG. 3, a needle assembly designed according to this invention is readily adaptable to a wide variety of conventional syringe styles.

FIGS. 4 and 5 illustrate an alternative embodiment of an intradermal delivery device 80 that includes a syringe made from two sheets of thermoplastic material. The syringe includes a body portion 82 that is generally flat and surrounds a reservoir 84. An outlet port 86 allows fluid substance within the reservoir 84 to be communicated out of the reservoir to administer an injection. The syringe body preferably is formed using a thermoforming process as is known in the art.

A receiver 90 includes a generally cylindrical neck portion 92 that preferably is secured to the outlet port 86 using a heating or welding process as is known in the art. A flange 94 preferably rests against the body portion 82 of the syringe to provide structural integrity. An extension 96 extends away from the flange 94 in a direction opposite from the cylindrical portion 92. The needle assembly 20 preferably is received within the extension 96 as shown in FIG. 5.

The receiver 90 preferably supports a sealing membrane 100 that closes off the outlet port 86 so that they syringe can be prefilled. The needle assembly 20 preferably includes a back end 102 of the needle that penetrates the sealing membrane 100 when the hub 22 is received within the extention 96.

The side walls of the reservoir 84 preferably are squeezed between a thumb and index finger so that the side walls collapse towards each other and the substance within the reservoir 84 is expelled through the opening in the forward end 40 of the needle 24. In the embodiment of FIGS. 4 and 5, the hub 22 and limiter 26 preferably are integrally moduled as a single piece of plastic material. A snap fit arrangement secures the hub 22 within the extension 96 of the receiver 90. Another alternative is illustrated in FIGS. 6 and 7. In this embodiment, the hub 22 is molded separately from the limiter 26, which is integrated with the extension 96. A difference between the embodiments of FIGS. 6 and 7 compared to that of FIGS. 4 and 5 includes an elongated extension 96 so that the side wall of the extension 96 provides the skin engaging surface 42 of the limiter 26. In this embodiment, the limiter is supported by the syringe body. By appropriately choosing the dimensions of the needle 24 and the length of the extension 96, the desired distance d between the skin engaging surface 42 and the needle tip 40 can be achieved.

FIG. 7 also illustrates a needle shield 110, which preferably is provided on the hub 22 and needle 24. The needle shield 110 facilitates inserting the hub 22 within the receiver 90 until the hub 22 is appropriately received within the extension 96 so that the intradennal delivery device 80 is ready for use. The needle shield 110 can be discarded after the hub 22 is in position. Alternatively, the needle shield 110 can be replaced over the needle 24 after an injection is complete to avoid the possibility for a needle stick while handling the intradermal delivery device 80 after it has been used. Although the shield 110 is only shown in FIG. 7, it preferably is utilised with the embodiment of FIGS. 4-7.

This invention provides an intradermal needle injector that is adaptable to be used with a variety of syringe types. Therefore, this invention provides the significant advantage of facilitating manufacture and assembly of intradermal needles on a mass production scale in an economical fashion.

Operation and Use

Having described the preferred embodiments of the intradermal delivery device 80 of the present, including the needle assembly 20 and drug container 60, its operation and use is described below.

Use of the delivery device to administer substances vaccines into the intradermal layer is significantly easier than with a traditional syringe and needle. Using a traditional syringe and needle is technique-dependent and requires considerable skill to develop an acceptable skin wheal. In particular, the needle must be carefully guided at a shallow angle under the skin while maintaining correct orientation of the needle bevel. In constrast, with a prefilled intradermal delivery device of the present invention, the user simply presses the device perpendicularly on to the skin and injects the substance. The depth of penetration of the needle is mechanically limited to the intradermal space. In this way, there is no need to orient the needle bevel during injection. Orienting the device, particularly the needle, perpendicularly to the skin, as well as stability while injecting the substance, is facilitated by the design of the device.

Referring now to FIG. 8, an example method of filling devices designed according to this invention is schematically illustrated in flow chart format. When the device includes a syringe of the style illustrated in FIG. 3, the following basic procedure is useful for pre-filling the syringes with a desired substance.

A supply of syringe barrels 200 includes the desired form of syringe, such as those illustrated and discussed above. A locally controlled environment 202 preferably is maintained in a known manner. The locally controlled environment 202 preferably is situated to immediately accept the syringes without requiring any intermediate cleaning or sterilising steps between the supply 200 and the environment 202.

In one example, the syringe barels are washed with air at 204 to remove any particulates from the syringes. The syringes preferably are then coated at 206 with a lubricant such as a lubricating silicone oil on the inner surface. The lubricant facilitates moving the stopper 70 and plunger 66 through the syringe during actual use of the device.

The end of syringes that eventually will need assembly 20 may be capped with a tip cap within the environment 202. In one example, tip caps are supplied at 208. The tip caps are air washed at 210. The cleaned tip caps and syringe barrels are conveyed to an assembly device 212 where the tip caps are secured onto the syringes. The syringe barrel assemblies are then conveyed to a filling station 214 to be filed with the desired substance.

Once filled as desired, the stoppers 70 are inserted into the open end of the syringes at 220. Prior to inserting the stoppers 70, they preferably are assembled with the plunger rods 66 at 222 and lubricated at 224 with a conventional lubricant in a known manner. The assembled, filled syringes preferably are inspected at 226 for defects and discharged from the locally controlled environment.

The syringes typically will be sterilised at 230 and packaged at 232 into individual packages or into bulk packaging depending on the needs of a particular situation. Suitable sterilisation techniques are known and will be chosen by those skilled in the art depending on the needs of a particular situation or to accommodate the properties of a given substance. Sterilising a device designed according to this invention can be completed before or after packaging.

Variations of the filling steps are within the scope of this invention. For example, the stopper can be inserted first, then fill the syringe, followed by applying a tip cap. Additionally, when the device includes a syringe body of the type shown in FIGS. 4 and 5, for example, the filling operation obviously does not include insertion of a stopper nor the lubrication steps described above. Instead, appropriate filling techniques that are known are utilised.

The actual insertion of the desired substance into the syringe body can be accomplished in any of several known manners. Example filling techniques are disclosed in U.S. Pat. No. 5,620,425 to Hefferman et al.; U.S. Pat. No. 5,597,530 to Smith et al.; U.S. Pat. No. 5,537,042 to DeHaen; U.S. Pat. No. 5,531,255 to Vacca; U.S. Pat. No. 5,519,984 to Veussink et al.; U.S. Pat. No. 5,373,684 to Veussink et al.; U.S. Pat. No. 5,265,154 to Liebert et al.; U.S. Pat. No. 5,287,983 to Liebert et al.; and U.S. Pat. No. 4,718,463 to Jurgens, Jr. et al., each of which is incorporated by reference into this application.

The Flu vaccine of the present invention will now be further described with reference to the following non limiting Examples.

### EXAMPLES

### Example 1

### Preparation of Split Influenza Vaccine

Each strain for the split vaccine was prepared according to the following procedure.

Preparation of Virus Inoculum

On the day of inoculation of embryonated eggs a fresh inoculum is prepared by mixing the working seed lot with a phosphate buffered saline containing gentamycin sulphate at 0.5 mg/ml and hydrocortisone at 25 μg/ml. (virus strain-dependent). The virus inoculum is kept at 2-8° C.

Inoculation of Embryonated Eggs

Nine to eleven day old embryonated eggs are used for virus replication. Shells are decontaminated. The eggs are inoculated with 0.2 ml of the virus inoculum. The inoculated eggs are incubated at the appropriate temperature (virus strain-dependent) for 48 to 96 hours. At the end of the incubation period, the embryos are killed by cooling and the eggs are stored for 12-60 hours at 2-8° C.

Harvest

The allantoic fluid from the chilled embryonated eggs is harvested. Usually, 8 to 10 ml of crude allantoic fluid is collected per egg. To the crude monovalent virus bulk 0.100 mg/ml thiomersal is optionally added.

Concentration and Purification of Whole Virus from Allantoic Fluid

1. Clarification

The harvested allantoic fluid is clarified by moderate speed centrifugation (range: 4000-14000 g).

2. Adsorption Step

To obtain a CaHPO₄ gel in the clarified virus pool, 0.5 mol/L Na₂HPO₄ and 0.5mol/L CaCl₂ solutions are added to reach a final concentration of CaHPO₄ of 1.5 g to 3.5 g CaHPO₄/litre depending on the virus strain.

After sedimentation for at last 8 hours, the supernatant is removed and the sediment containing the influenza virus is resolubilised by addition of a 0.26 mol/L EDTA-Na₂ solution, dependent on the amount of CaHPO₄ used.

3. Filtration

The resuspended sediment is filtered on a 6 μm filter membrane.

4. Sucrose Gradient Centrifugation

The influenza virus is concentrated by isopycnic centrifugation in a linear sucrose gradient (0-55% (w/v)) containing 100 μg/ml Thiomersal. The flow rate is 8-15 litres/hour.

At the end of the centrifugation, the content of the rotor is recovered by four different fractions (the sucrose is measured in a refractometer):

| | |
|---|---|
| fraction 1 | 55-52% sucrose |
| fraction 2 | approximately 52-38% sucrose |
| fraction 3 | 38-20% sucrose* |
| fraction 4 | 20-0% sucrose |

*virus strain-dependent: fraction 3 can be reduced to 15% sucrose.

For further vaccine preparation, only fractions 2 and 3 are used.

Fraction 3 is washed by diafiltration with phosphate buffer in order to reduce the sucrose content to approximately below 6%. The influenza virus present in this diluted fraction is pelleted to remove soluble contaminants.

The pellet is resuspended and thoroughly mixed to obtain a homogeneous suspension. Fraction 2 and the resuspended pellet of fraction 3 are pooled and phosphate buffer is added to obtain a volume of approximately 40 litres. This product is the monovalent whole virus concentrate.

5. Sucrose Gradient Centrifugation with Sodium Deoxycholate

The monovalent whole influenza virus concentrate is applied to a ENI-Mark II ultracentrifuge. The K3 rotor contains a linear sucrose gradient (0-55% (w/v)) where a sodium deoxycholate gradient is additionally overlayed. Tween 80 is present during splitting up to 0.1% (w/v). The maximal sodium deoxycholate concentration is 0.7-1.5% (w/v) and is strain dependent. The flow rate is 8-15 litres/hour.

At the end of the centrifugation, the content of the rotor is recovered by three different fractions (the sucrose is measured in a refractometer) Fraction 2 is used for further processing. Sucrose content for fraction limits (47-18%) varies according to strains and is fixed after evaluation:

6. Sterile Filtration

The split virus fraction is filtered on filter membranes ending with a 0.2 μm membrane. Phosphate buffer containing 0.025% (w/v) Tween 80 is used for dilution. The final volume of the filtered fraction 2 is 5 times the original fraction volume.

7. Inactivation

The filtered monovalent material is incubated at 22±2° C. for at most 84 hours (dependent on the virus strains, this incubation can be shortened). Phosphate buffer containing 0.025%

Tween 80 is then added in order to reduce the total protein content down to max. 250 μg/ml. Formaldehyde is added to a final concentration of 50 μg/ml and the inactivation takes place at 20° C.±2° C. for at least 72 hours.

8. Ultrafiltration

The inactivated split virus material is concentrated at least 2 fold in a ultrafiltration unit, equipped with cellulose acetate membranes with 20 kDa MWCO. The Material is subsequently washed with phosphate buffer containing 0.025% (w/v) Tween 80 and following with phosphate buffered saline containing 0.01% (w/v) Tween.

9. Final Sterile Filtration

The material after ultrafiltration is filtered on filter membranes ending with a 0.2 μm membrane. The final concentration of Haemagglutinin, measured by SRD (method recommended by WHO) should exceed 450 μg/ml.

10. Storage

The monovalent final bulk is stored at 2-8° C. for a maximum of 18 months.

Purity

Purity was determined semiquantitatively by O.D. scanning of Coomassie-stained polyacrylamide gels. Peaks were determined manually. Sample results are given in Table 1.

TABLE 1

| | | | | | Other viral and |
|---|---|---|---|---|---|
| Viral Proteins (HA, NP, M) % | | | | | host-cell derived |
| H3N2 | HA dimer | HA1 + 2 | NP | M | proteins % |
| A/Syd/5/97 | 10.34 | 22.34 | 25.16 | 37.33 | 4.83 |
| A/Nan933/95 | 8.17 | 15.8 | 40.09 | 30.62 | 5.32 |
| B | | | | | |
| B/Har/7/94 | 5.71² | 24.07 | 15.64 | 50 | 4.58 |
| B/Yam/166/98 | 0.68 | 27.62 | 21.48 | 46.02 | 4.2 |
| H1N1 | | | | | |
| A/Tex/36/91 | | 33.42 | 24.46 | 34.33 | 7.79 |
| A/Bei/262/95 | | 32.73 | 35.72 | 27.06 | 4.49 |
| H2N2 | | | | | |
| A/sing/1/57 | 2.8 | 39.7 | 21.78 | 32.12 | 3.6 |

A particular combination of strains for use in the invention includes A/New Caledonia/20/99 (H1N1), A/Panama/20/99 (H3N2) and B/Yamanashi/166/98.

### Example 2

### Preparation of Vaccine Doses from Bulk Vaccine

Final vaccine is prepared by formulating a trivalent vaccine from the monovalent bulks with the detergent concentrations adjusted as required.

PBS, pH 7.2±0.2, Tween 80 and Triton X-100 are mixed to obtain the required final concentrations (PBS 1× concentrated, Tween 80 0.15% and Triton X-100 0.02%) . The three following inactivated split virions are added with 10 minutes stirring in between:
15 μg A/New Caledonia/20/99 (H1N1)
15 μg A/Panama/20/99 (H3N2)

15 μg B/Yamanashi/166/98

After 15 minutes stirring pH is adjusted to 7.2±0.2.

The dose volume is 500 μl. The doses are filled in sterile ampoules. Immediately before applying the vaccine, 0.1 ml doses are removed from the ampoule using the device for intradermal application.

### Example 3

### Methods used to Measure Antibody Responses

1. Detection of Specific Anti-Flu and Total IgA in Human Nasal Secretions by ELISA

Collection Method for Human Nasal Secretions

An appropriate method is used to collect nasal secretions, for example a classical nasal wash method or a nasal wick method.

After collection and treatment of human nasal secretions, the detection of total and specific anti-FLU IgA is realized with ELISAs e.g:

Capture ELISA for Detection of Total IgA

Total IgA are captured with anti-human IgA polyclonal affinity purified Ig immobilized on microtiter plates and subsequently detected using a different polyclonal anti-human IgA affinity purified Ig coupled to peroxidase.

A purified human sIgA is used as a standard to allow the quantification of sIgA in the collected nasal secretions.

3 references of purified human sIgA are used as low, medium and high references in this assay.

Direct ELISA for Detection of Specific Anti-FLU IgA

Three different ELISAs are performed, one on each FLU strain present in the vaccine formulation.

Specific anti-FLU IgA are captured with split inactivated FLU antigens coated on microtiter plates and subsequently detected using the same different polyclonal anti-human IgA affinity purified Ig coupled to peroxidase as the one used for the total IgA ELISA.

Results—Expression and Calculations

Total IgA Expression

The results are expressed as μg of total IgA in 1 ml of nasal fluids, using a Softmaxpro program.

Specific Anti-Flu IgA Expression

The results are expressed as end-point unit titer, which are calculated as the inverse of the last dilution which gives an OD₄₅₀ₙₘ above the cut off.

The final results of a sample are expressed as follows:

Normalization of the specific response by calculating the ratio between the specific response and the total IgA concentration: end-point unit/pg total IgA ( most commonly used calculation method in the literature).

2. Haemagglutination Inhibition (HAI) Activity of Flu-Specific Serum Abs

Sera (50 μl) are treated with 200 μl RDE (receptor destroying enzyme) for 16 hours at 37° C. The reaction is stopped with 150 μl 2.5% Na citrate and the sera are inactivated at 56° C. for 30 min. A dilution 1:10 is prepared by adding 100 μl PBS. Then, a 2-fold dilution series is prepared in 96 well plates (V-bottom) by diluting 25 μl serum (1:10) with 25 μl PBS. 25 μl of the reference antigens are added to each well at a concentration of 4 hemagglutinating units per 25 μl. Antigen and antiserum dilution are mixed using a microtiter plate shaker and incubated for 60 minutes at room temperature. 50 μl chicken red blood cells (RBC) (0.5%) are then added and the RBCs are allowed to sediment for 1 hour at RT. The HAI titre corresponds to the inverse of the last serum dilution that completely inhibits the virus-induced hemagglutination.

1. An intradermal delivery device for the intradermal delivery of a flu vaccine, the device comprising:
i a container comprising a flu vaccine and having an outlet port;
ii a needle in fluid communication with the outlet port, the needle having a forward end that is adapted to penetrate skin; and
iii a limiter that surrounds the needle and has a skin engaging surface that is adapted to be received against the skin to receive an intradermal injection, the needle forward end extending beyond the skin engaging surface a selected distance such that the limiter portion limits an amount that the needle is able to penetrate through the skin.

2. The device of claim 1, wherein the drug container is a syringe including a generally hollow, cylindrical body portion and a plunger that is received within the reservoir, the plunger being selectively movable within the reservoir to cause the substance to be forced out of the outlet port during an injection.

3. The device of claim 1, including a hub portion that supports the needle and the hub portion is selectively secured to the drug container near the outlet port.

4. The device of claim 1, wherein the drug container is a syringe having a resevoir adapted to contain the vaccine, the syringe including a generally flat body portion that at least partially surrounds the reservoir, the body portion and the reservoir being made from two sheets of thermoplastic material such that side wails of the reservoir are selectively deflected toward each other to expel a substance from the reservoir during an injection.

5. The device of claim 4, including a hub that supports the needle and is selectively secured to the syringe near the outlet port and a receiver adjacent the outlet port that is generally circular and the hub is completely received within the receiver and wherein the limiter is integrally formed with the receiver such that the limiter is permanently supported by the body portion adjacent the outlet port.

6. The device of claim 5, wherein the skin engaging surface surrounds the needle, and has a thickness defined between an inner diameter and an outer diameter and wherein the inner diameter is at least five times greater than an outside diameter of the needle.

7. The device of claim 6, wherein the skin engaging surface is generally circular.

8. The device of claim 5, wherein the needle forward end extends away from the hub in a first direction and a needle back end extends away from the hub in a second direction, and including a sealing membrane that closes off the outlet port and wherein the needle back end pierces the sealing membrane when the hub is received by the receiver.

9. The device of claim 4, including a hub that supports the needle and is selectively secured to the syringe near the outlet port and a receiver adjacent the outlet port that is generally circular and the hub is completely received within the receiver and wherein the limiter is formed separately from the receiver and is at least partially received by the receiver.

10. The device of claim 9, wherein the limiter and the hub are integrally formed into a single piece structure.

11. The device of claim 1, wherein the needle has a length and wherein the selected distance is much less than the needle length.

12. The device of claim 11, wherein the selected distance is fixed and is in the range from approximately 0.5 mm to approximately 3 mm.

13. The device of claim 1, wherein the skin engaging surface is generally flat and extends through a plane that is generally perpendicular to an axis of the needle.

14. The device of claim 1, wherein the skin engaging surface includes a central opening that is slightly larger than an outside dimension of the needle and the skin engaging surface is continuous.

15. The device of claim 1, wherein the skin engaging surface includes a contact surface area that is large enough to stabilise the assembly in a desired orientation relative to the skin.

16. The device of claim 1, wherein the desired orientation is generally perpendicular to the skin.

17. The device of claim 1, wherein the drug container is pre-filled with a substance.

18. A kit for use in intradermal flu vaccine delivery comprising:
i a vaccine container comprising a flu vaccine and
ii a hypodermic needle assembly, the assembly comprising:
a hub portion that is able to be attached to a drug container;
a needle supported by the hub portion, the needle having a hollow body with a forward end extending away from the hub portion; and
a limiter portion that surrounds the needle and extends away from the hub portion toward the forward end of the needle, the limiter portion having a skin engaging surface that is adapted to be received against the skin of an animal to receive an intradermal injection, the needle forward end extending beyond the skin engaging surface a selected distance such that the limiter portion limits an amount that the needle is able to penetrate through the skin of an animal.

19. The kit according to claim 18, wherein the hub portion and the limiter portion are integrally formed as a single piece made from a plastic material.

20. The kit according to claim 18, wherein wherein the hub portion and the limiter portion are formed as separate pieces.

21. The kit according to claim 20, wherein the limiter portion includes an inner cavity that receives at least a portion of the hub portion and the inner cavity includes an abutment surface that engages corresponding structure on the hub portion to thereby limit the amount that the needle forward end extends beyond the skin engaging surface.

22. The kit according to claim 20, wherein the limiter portion is integrally formed as part of the syringe and the hub portion is received within the limiter portion.

23. The kit according to claim 22, wherein the skin engaging surface surrounds the needle, and has a thickness defined between an inner diameter and an outer diameter and wherein the inner diameter is at least five times greater than an outside diameter of the needle.

24. The kit according to claim 23, wherein the skin engaging surface is generally circular.

25. The kit according to claim 18, wherein the skin engaging surface includes a central opening that is slightly larger than an outside diameter of the needle and the skin engaging surface is continuous.

26. The kit according to claim 18, wherein the skin engaging surface is generally flat and extends through a plane that is generally perpendicular to an axis of the needle.

27. The kit according to claim 18, wherein the selected distance that the forward end of the needle extends beyond the skin engaging surface is fixed.

28. The kit according to claim 18, wherein the selected distance is in the range from approximately 0.5 mm to approximately 3 mm.

29. The kit according to claim 18, wherein the skin engaging surface includes a contact surface area that is large enough to stabilise the assembly in a desired orientation relative to the skin.

30. The kit according to claim 29, wherein the desired orientation is generally perpendicular to the skin.

31. The kit according to claim 18, wherein the drug container is a syringe and the animal is human.

32. A device according to any of claims, or a kit according to any of claims 1-31, wherein the flu vaccine is obtainable by the following process:
(i) harvesting of virus-containing material from a culture;
(ii) clarification of the harvested material to remove non-virus material;
(iii) concentration of the harvested virus;
(iv) a further step to separate whole virus from non-virus material;
(v) splitting of the whole virus using a suitable splitting agent in a density gradient centrifugation step;
(vi) filtration to remove undesired materials;
wherein the steps are performed in that order but not necessarily consecutively.

33. A device or kit according to claim 32, wherein the intradermal flu vaccine is a trivalent non-live vaccine.

34. A device or kit according to claim 32, wherein the virus is grown on embryonated hen eggs and the harvested material is allantoic fluid.

35. A device or kit according to claim 32, wherein the clarification step is performed by centrifugation at a moderate speed.

36. A device or kit according to claim 32, wherein the concentration step employs an adsorption method such as CaHPO₄ adsorption.

37. A device or kit according to claim 32, wherein the further separation step (iv) is a zonal centrifugation separation using a sucrose gradient.

38. A device or kit according to claim 32, wherein the splitting step is performed in a further sucrose gradient, wherein the sucrose gradient contains the splitting agent.

39. A device or kit according to claim 38, wherein the splitting agent is sodium deoxycholate.

40. A device or kit according to claim 32, wherein the filtration step (vi) is an ultrafiltration step which concentrates the split virus material.

41. A device or kit according to claim 32, wherein there is at least one sterile filtration step, optionally at the end of the process.

42. A device or kit according to claim 32, wherein an inactivation step is performed prior to the final filtration step.

43. A device or kit according to claim 32, wherein the method comprises the further step of adjusting the concentration of one or more detergents in the vaccine composition.

44. A device or kit according to claim 32, wherein the vaccine is provided in a dose volume of between about 0.1 and about 0.2 ml.

45. A device or kit according to claim 32, wherein the vaccine is provided with an antigen dose of 1-5 μg haemagglutinin per strain of influenza present.

46. A device or kit according to claim 32, wherein the vaccine meets the EU criteria for at least two strains.

47. A device or kit according to claim 32, wherein the vaccine further comprises a bile acid or cholic acid, or derivative thereof such as sodium deoxycholate.

48. A device or kit according to claim 32, wherein the vaccine comprises at least one non-ionic surfactant.

49. A device or kit according to claim 32, wherein the at least one non-ionic surfactant selected from the group consisting of the octyl- or nonylphenoxy polyoxyethanols (for example the commercially available Triton™ series), polyoxyethylene sorbitan esters (Tween™ series) and polyoxyethylene ethers or esters of general formula (I):
HO(CH₂CH₂O)ₙ-A-R (I)
wherein n is 1-50, A is a bond or —(O)—, R is C₁₋₅₀ alkyl or phenyl C₁₋₅₀ alkyl; and combinations of two or more of these.

50. A device or kit according to claim 49, wherein the vaccine comprises a combination of polyoxyethylene sorbitan monooleate (Tween 80) and t-octylphenoxy polyethoxyethanol (Triton X-100).
